(19) United States
(12) Patent Application Publication
(10) Pub. No.: US 2024/0091541 A1
(43) Pub. Date: Mar. 21, 2024

(54) SYSTEMS AND DEVICES FOR CARDIAC ARRHYTHMIA THERAPIES

(71) Applicant: MAXWELL BIOMEDICAL INC., San Diego, CA (US)

(72) Inventors: Sumeet DHAM, Plymouth, MN (US); David W. BOURN, Champlin, MN (US); Jeffrey PETERS, Excelsior, MN (US); Patrick MILLER, Rogers, MN (US)

(21) Appl. No.: 18/038,526

(22) PCT Filed: Nov. 30, 2021
(86) PCT No.: PCT/US2021/061257

§ 371 Date: May 24, 2023

Related U.S. Application Data
(63) Continuation of application No. 17/472,317, filed on Sep. 10, 2021, now abandoned.
(60) Provisional application No. 63/119,970, filed on Dec. 1, 2020.

(51) Int. Cl. A61N 1/362; A61N 1/368; A61N 1/37

(52) U.S. Cl. CPC A61N 1/3622; A61N 1/3624; A61N 1/3684; A61N 1/3702

(57) ABSTRACT

A system for treating cardiac arrhythmias comprising a generator including: a sensing circuitry configured to evaluate one or more identified signals representative of electrical activity of the heart and detect an arrhythmia, a control circuitry that is configured to control delivery of a therapy in response to the detected arrhythmia, the therapy including a first stage of electrical pulses delivered via at least a first electrode, wherein the first set of electrical pulses is configured to destabilize and/or terminate a reentry associated with the arrhythmia, and a first lead coupled to the generator, wherein the first lead includes the first electrode. The therapy further includes a second stage of electrical pulses delivered via the first electrode and/or a second electrode, wherein the second stage of pulses is configured to terminate the reentry; and an inter-stage delay separating the first and second stages of electrical pulses. A second lead is in communication with the control circuitry, wherein the second lead includes the second electrode.

### CROSS-REFERENCE TO RELATED APPLICATIONS

This application claims the benefit of priority from U.S. application Ser. No. 17/472,317, filed on Sep. 10, 2021, and U.S. Provisional Application No. 63/119,970, filed on Dec. 1, 2020, both of which are incorporated by reference herein in their entireties.

### TECHNICAL FIELD

The present disclosure relates generally to systems, device, and methods for the treatment of cardiac arrhythmias, such as atrial arrhythmia and/or ventricular arrhythmia. More particularly, the present disclosure relates to systems, devices, and methods of using electrical stimuli from an implantable device that delivers an antiarrhythmic therapy to destabilize and extinguish reentry mechanisms that maintain the cardiac arrhythmia. The disclose systems, devices, and methods may be suitable for treating atrial arrhythmias only, ventricular arrhythmias only, and/or both atrial and ventricular arrhythmias.

### BACKGROUND

Cardiac arrhythmia refers to a change in the normal sequence of electrical impulses that coordinates the regular beating of the heart, resulting in the heart to beat irregularly, too slowly, or too quickly. While cardiac arrhythmias may be harmless in some instances, cardiac arrhythmias may also cause bothersome, or even life-threatening, conditions in many individuals. Examples of cardiac arrhythmias include atrial arrhythmias, which refers to arrhythmias in the atria, i.e., the heart's upper chambers, and ventricular arrhythmias, which refers to arrhythmias in the ventricles, i.e., the heart's lower chambers.

Atrial tachyarrhythmias are the most common atrial arrhythmia. There are two primary forms of atrial tachyarrhythmias—atrial fibrillation (AF) and atrial flutter (AFl)— with relative occurrence in their chronic forms of about 10:1, respectively. Many different factors can promote the initiation and maintenance of AF and AFl. Several cardiac disorders may predispose patients to AF, including coronary artery disease, pericarditis, mitral valve disease, congenital heart disease, congestive heart failure (CHF), thyrotoxic heart disease, and hypertension. Many of these are thought to promote AF by increasing atrial pressure and/or causing atrial dilation. AF also occurs in individuals without any evidence of heart or systemic disease, a condition known as “lone AF,” which primarily involves the autonomic nervous system.

Both AF and AFl are maintained by a reentry mechanism. Specifically, atrial tissue continually excites itself, creating a reentrant, i.e., circular or tornado-like patterns of excitation. AFl is generally defined as a macro-reentrant circuit, which can rotate around a functional or anatomic line of block. Major anatomical structures are usually involved in defining one or several simultaneous reentry circuit(s), including the region between superior and inferior venae cavae in the right atrium, and the pulmonary vein region in the left atrium. If the cycle length (“CL”) of the reentry remains relatively long, one-to-one conduction can remain throughout the entire atria and AFl can be observed. However, if the CLs of reentry circuits are sufficiently short, waves of excitation produced by the reentrant circuit break up in the surrounding atrial tissue, and AF can ensue. The morphology of electrograms during AFl or AF depends on the anatomic location and frequency of reentrant circuits that cause the arrhythmia.

There are clear interactions between AF and AFl. AFl is defined as the presence of a single, constant, and stable reentrant circuit. AF, on the other hand, may be due to random activation in which multiple reentrant wavelets of the leading circle type (mother rotor) continuously circulate in directions determined by local excitability, refractoriness, and anatomical structure. AF may be converted to AFl, and vice versa, spontaneously or as a result of an intervention, such as drug administration, direct current cardioversion (DCCV), or atrial pacing.

AF is the most prevalent clinical arrhythmia and, with an aging population, has the potential of becoming an increasing cause of morbidity and mortality. Although several options for pharmaceutical treatment exist, for some patients, particularly those with paroxysmal AF, drug therapies may be ineffective. In addition, anti-arrhythmic drugs may cause serious proarrhythmic side effects. An alternative to pharmacological treatment of AF is a cardiac ablation procedure. While there have been many advances in ablative techniques, these procedures are not without risks. Such risks can include cardiac perforation, esophageal injury, embolism, phrenic nerve injury, and/or pulmonary vein stenosis.

Another alternative to pharmacological treatment of atrial tachyarrhythmias may be electrical stimulation therapies. Such therapies are currently limited to DCCV, i.e., external cardioversion, internal cardioversion, and a device-based therapy called atrial antitachycardia pacing (aATP). It is noted that DCCV involves externally applied shocks that necessarily recruit more of the skeletal musculature, which may result in heightened pain and discomfort to patients, and internal cardioversion is not always a practical avenue of therapy as it requires a catheterization laboratory setting.

Meanwhile, aATP refers to the delivery of a burst of pacing stimuli at an empirically chosen frequency at a single pacing site in order to stimulate the excitable gap of a reentrant circuit, and disrupt and terminate the circuit. aATP may be delivered via near-field electrodes of an implantable device, e.g., a tip-ring electrode combination from a right atrial lead. Although aATP may be effective for slower AFls, the effectiveness of aATP may diminish for CLs below about two hundred milliseconds (“ms”) and can be ineffective for faster AFl and AF. aATP failure may occur when the pacing lead of an implanted device is located at a distance from the reentrant circuit and there is no excitable pathway for the pacing induced wavefront to penetrate the reentrant circuit. This may be a highly probable scenario for faster arrhythmias. In addition, the application of other atrial anti-tachycardia therapies may potentially induce ventricular fibrillation, as described, for example, in U.S. Pat. No. 6,091,991 to Warren; U.S. Pat. No. 6,847,842 to Rodenhiser et al.; U.S. Pat. No. 7,110,811 to Wagner et al.; and U.S. Pat. No. 7,120,490 to Chen et al, all of which are incorporated herein by reference.

Ventricular arrhythmias, including ventricular tachycardia (VT) and ventricular fibrillations (VF), may be formed in a similar manner as discussed above for atrial arrhythmias. Rotating waves of electrical activity or reentrant circuits, e.g., 1) functional reentries, which involve freely rotating waves and 2) anatomical reentries, where a wave rotates around an obstacle such as a blood vessel or piece of ischemic tissue, are also a factor in forming VT or VF.

Current VT/VF electrical stimulation therapies face similar issues as those described above for AF therapies. For example, traditional defibrillation, e.g., external or internal cardioversion, may not be a preferred means of therapy as defibrillation involves the use of high voltage shocks, which may have undesirable side effects. Antitachycardia pacing (ATP) in the ventricles suffers from the same limitations as aATP and may also be ineffective in treating VT, as ATP may not be as effective against anatomical high frequency reentries.

Consequently, there remains a need for improved electrical stimulation therapies to treat both atrial and ventricular tachyarrhythmias.

### SUMMARY OF THE DISCLOSURE

According to an example, a system for treating cardiac arrhythmias may comprise a generator including a sensing circuitry configured to evaluate one or more identified signals representative of electrical activity of the heart and detect an arrhythmia, a control circuitry that is configured to control delivery of a therapy in response to the detected arrhythmia, the therapy including a first stage of electrical pulses delivered via at least a first electrode, wherein the first set of electrical pulses is configured to destabilize and/or terminate a reentry associated with the arrhythmia, and a first lead coupled to the generator, wherein the first lead includes the first electrode.

In another example, the system may further comprise a second lead coupled to the generator, wherein the second lead includes a second electrode, and wherein the therapy may further include a second stage of electrical pulses delivered via at least the first electrode and/or the second electrode, wherein the second stage of pulses is configured to terminate the reentry. The first lead or the second lead may further include a third electrode, and the therapy further includes a third stage of electrical pulses delivered between the third electrode and the first electrode or the second electrode, wherein the third stage of pulses is configured to terminate the reentry. The therapy may further include a first inter-stage delay between the first stage and the second stage, the first inter-stage delay being between 50 ms to 300 ms. The therapy may further include a second inter-stage delay between the second stage and the third stage, the second inter-stage delay being between 50 ms to 300 ms. The first stage of electrical pulses may be delivered as far-field electrical stimulation, the second stage of electrical pulses is delivered as far-field electrical stimulation, and the third stage of electrical pulses is delivered as near-field electrical stimulation. The first stage of electrical pulses may be biphasic. Alternative, the first stage of electrical pulses may be monophasic. The second stage of electrical pulses may be monophasic and the third stage of electrical pulses may be monophasic. The generator may further include a first connector coupled to a first end of the first lead, and a second connector coupled to a first end of the second lead. The first connector may be a DF-4 connector. The second connector may be an IS-4 or a DF-4 connector. Alternatively, the second connector is an IS-1 connector.

According to an example, a system for treating cardiac arrhythmias may comprise a first circuitry configured to evaluate one or more identified signals representative of electrical activity of the heart and detect an arrhythmia, a second circuitry that is configured to control delivery of a therapy in response to the detected arrhythmia, the therapy including a first stage of electrical pulses delivered via at least a first electrode, wherein the first stage of electrical pulses is configured to destabilize and/or terminate a reentry associated with the arrhythmia, a second stage of electrical pulses delivered via the first electrode and/or a second electrode, wherein the second stage of pulses is configured to terminate the reentry, and an inter-stage delay separating the first and second stages of electrical pulses, a first lead in communication with the second circuitry, wherein the first lead includes the first electrode, and a second lead in communication with the second circuitry, wherein the second lead includes the second electrode.

In an example, the first stage may be delivered between the first electrode and the second electrode, and the second stage is delivered between the first electrode and the second electrode. The first lead or the second lead may further include a third electrode, and the therapy may further include a third stage of electrical pulses delivered between the third electrode and the first electrode or the second electrode. The third electrode may comprise a tip ring. The first electrode may comprise a far-field electrode, the second electrode may comprise a far-field electrode, and the third electrode may comprise a near-field electrode. The first lead may be configured to be inserted within a coronary sinus so that a first portion of the first lead is adjacent to a left atrium and a second portion of the first lead is adjacent to a left ventricle. The second lead may be configured to extend within a heart so that a portion of the second lead is adjacent to a right atrium of the heart. The second lead may be configured to extend within a heart so that a portion of the second lead is adjacent to a right ventricle of the heart. The first lead may be further configured to deliver a Cardiac Resynchronization Therapy (CRT).

According to an example, a method for treating cardiac arrhythmia may comprise identifying irregular electrical activity indicative of an arrhythmia, and in response to identifying the arrhythmia, delivering a therapy to the heart via a plurality of electrodes, the therapy including a first stage of electrical pulses delivered via at least a first electrode of the plurality of electrodes, the first stage of electrical pulses configured to destabilize and/or terminate a reentry associated with the arrhythmia, wherein the first stage of electrical pulses is delivered as far-field electrical stimulation.

In another example, the method may further comprise a second stage of electrical pulses delivered via the first electrode and/or a second electrode of the plurality of electrodes, the second stage of pulses configured to terminate the reentry, and a first inter-stage delay separating the first and second stages of electrical pulses, wherein the second stage of electrical pulses is delivered as far-field electrical stimulation or near-field. The method may further comprise a third stage of electrical pulses delivered via at least a third electrode of the plurality of electrodes, and a second inter-stage delay separating the second and third stages of electrical pulses, wherein the second stage of electrical pulses is delivered as far field electrical stimulation or near-field. The first stage of electrical pulses may be in biphasic waveform. Alternatively, the first stage of electrical pulses may be in monophasic waveform. The first stage of electrical pulses may include at least two pulses. Each of the pulses of the first stage may have a duration of less than or equal to 15 ms. The duration of the first stage of electrical pulses may be less than or equal to an S-T segment. Each of the pulses of the first stage may be delivered between 20 ms to 50 ms apart. Each of the pulses of the first stage may be delivered between 30 ms to 110 ms apart. The second stage of electrical pulses may be in monophasic waveform. The second stage of electrical pulses may include at least four pulses. Each of the pulses of the second stage may have a duration of 5 ms to 20 ms. Each of the pulses of the second stage May be delivered between 100 ms to 300 ms apart. The third stage of electrical pulses may be in monophasic waveform. The third stage of electrical pulses may include at least five pulses. Each of the pulses of the third stage may have a duration of 0.1 ms to 5 ms. Each of the pulses of the third stage may be delivered between 100 ms to 300 ms apart. The first inter-stage delay may be from 55 ms to 300 ms. The second inter-stage delay may be from 55 rips to 300 ms.

In another example, the plurality of electrodes may be positioned subcutaneously around the heart. The plurality of electrodes may be in direct contact with the heart via a leadless device. The plurality of electrodes may be in communication with a transmitter external to the patient, and the transmitter instructs the plurality of electrodes to deliver the therapy to the heart. The plurality of electrodes may be bioresorbable. Thus, the method may further comprise maintaining the plurality of electrodes within the patient, thereby allowing the plurality of electrodes to resorb and disappear without surgical extraction.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIGS. 1A-1B are diagrams of an exemplary medical system, according to some aspects of the present disclosure.

FIG. 1C is a perspective view of the medical system of FIGS. 1A-1B.

FIG. 2 is a diagram illustrating an exemplary atrial antiarrhythmic therapy, according to some aspects of the present disclosure.

FIG. 3 is a chart illustrating the atrial antiarrhythmic therapy of FIG. 2.

FIGS. 4-5 are respectively a diagram and a chart illustrating an exemplary atrial antiarrhythmic therapy, according to some aspects of the present disclosure.

FIGS. 6-7 are respectively a diagram and a chart illustrating an exemplary atrial antiarrhythmic therapy, according to some aspects of the present disclosure.

FIG. 8 is a perspective view of an exemplary medical system, according to some aspects of the present disclosure.

FIG. 9 is a perspective view of an exemplary lead of the medical system of FIG. 8.

FIGS. 10A-10B are perspective views of another exemplary lead of the medical system of FIG. 8.

FIG. 11 is an anterior view of a heart.

FIG. 12 is a diagram illustrating an exemplary ventricular antiarrhythmic therapy, according to some aspects of the present disclosure.

FIG. 13 is a chart illustrating the ventricular antiarrhythmic therapy of FIG. 12.

FIG. 14 is a perspective view of an exemplary medical system, according to some aspects of the present disclosure.

FIG. 15 is an anterior view of a heart.

FIG. 16 is a perspective view of another exemplary medical system, according to some aspects of the present disclosure.

FIG. 17 is a perspective view of another exemplary medical system, according to some aspects of the present disclosure.

FIG. 18 is a perspective view of another exemplary medical system, according to some aspects of the present disclosure.

### DETAILED DESCRIPTION

Reference will now be made in detail to aspects of the disclosure, examples of which are illustrated in the accompanying drawings. Wherever possible, the same or similar reference numbers will be used through the drawings to refer to the same or like parts. The term “distal” refers to a location or portion of a medical device farthest away from a user of the device, e.g., when introducing a device into a subject (e.g., patient). By contrast, the term “proximal” refers to a location or portion closest to the user, e.g., when placing the device into the subject.

Moreover, in the present disclosure, the term “near-field,” can relate to effects that are in close proximity to stimulating electrode(s), i.e., distances are restricted by several space constants (λ) of cardiac tissue, which is typically up to several millimeters. Near-field effects can be strongly dependent upon distance from the electrodes. The term “far-field,” on the other hand, can relate to effects that are generally independent or less dependent upon distance from the electrodes. They can occur at distances that are much greater than the space constant (λ).

Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. In this disclosure, relative terms, such as, for example, “about,” “substantially,” “generally,” and “approximately” are used to indicate a possible variation of ±10% in a stated value or characteristic.

This disclosure may solve one or more of the limitations in the art. The scope of the disclosure, however, is defined by the attached claims and not the ability to solve a specific problem. Embodiments of this disclosure include systems, devices, and methods for the treatment of atrial tachyarrhythmia and ventricular tachyarrhythmia.

An exemplary antiarrhythmic therapy may include at least a first stage for unpinning, and in some instances, also extinguishing, one or more singularities associated with an atrial or ventricular arrhythmia. Other exemplary antiarrhythmic therapies may further include a second stage for anti-repinning and/or extinguishing the one or more singularities associated with the atrial or ventricular arrhythmia, and/or a third stage for extinguishing of the one or more singularities associated with the atrial or ventricular arrhythmia. Thus, an exemplary antiarrhythmic therapy may be solely a first stage therapy, or any combination of a first stage, a second stage, and a third stage, as discussed further below. In some examples, the first stage may be a far-field therapy, the second stage may be a far-field therapy (or a far-field and near-field therapy), and the third stage may be a near-field therapy, but not limited thereto. It is further noted that, while the aforementioned first stage, second stage, and third stage therapies may be delivered sequentially, an antiarrhythmic therapy is not limited to such order. In other exemplary therapies, the first stage, second stage, and third stage therapies may be delivered in any order, e.g., third, first, and second, etc. It is noted that the parameters associated with each one of the aforementioned stages may depend on the type of antiarrhythmic therapy, e.g., atrial or ventricular, to be delivered, and are discussed in further detail below.

The above-noted antiarrhythmic therapy may be administered or delivered in response to a detected arrhythmia. Specifically, in some embodiments, the first stage of the therapy may be synchronized to an R-wave, i.e., the ventricular activation, and delivered within a ventricular refractory period.

The antiarrhythmic therapy may exploit a virtual electrode polarization (“VEP”), enabling successful treatment of atrial and ventricular tachyarrhythmia via the implantable treatment device. This may be enabled by far-field excitation of multiple areas of tissue, e.g., atrial or ventricular tissue, at once, rather than just one small area near a pacing electrode. Such far-field excitation may be effective for the treatment of atrial or ventricular tachyarrhythmia, as it may destabilize or terminate the core of a mother rotor, which anchors to myocardial heterogeneities such as a scar from myocardial infarction, the intercaval region, coronary arteries, or other fibrotic areas. This treatment may differ from conventional defibrillation therapy, which typically uses only one high-energy (about approximately 20 to approximately 40 joules) monophasic or biphasic shock or two sequential monophasic shocks from two different vectors of far-field electrical stimuli.

Applying far-field low energy electric field excitation/stimulation in an appropriate range of time-and frequency-domains may interrupt and terminate a reentrant circuit by selectively depolarizing (exciting) and hyperpolarizing (de-exciting) areas near the core of reentry. By stimulating the areas near the core of the circuit, the reentry may be disrupted and terminated. The reentrant circuit may be anchored at a functionally or anatomically heterogeneous region, which constitutes the core of reentry. Areas near the heterogeneous regions (including the region of the core of reentry) will experience greater polarization in response to an applied electric field compared with the surrounding, more homogeneous tissue. Thus, the region near the core of reentry may be preferentially excited with very small electric fields to destabilize or terminate anchored reentrant circuits. Once destabilized, subsequent stimulation, while not necessary, may terminate the arrhythmia and restore normal sinus rhythm.

Virtual electrodes occur proximal to heterogenous areas including local resistive heterogeneities to depolarize a critical part of the reentry pathway or excitable gap near the core of reentry. As noted above, various pulse (stage) protocols for an antiarrhythmic therapy to terminate atrial/ventricular arrhythmias in accordance with aspects of the present disclosure are contemplated. In one aspect, the reentry is either terminated directly or destabilized by far-field pulses delivered in a first stage and/or second stage, and/or terminated by additional stimuli by near-field pulses delivered in a third stage of the antiarrhythmic therapy.

When a voltage shock is applied to a cell membrane of the heart, the membrane does not respond to the shock immediately. The cell response lags behind the voltage applied via the electrode(s). When the applied voltage comprises a biphasic pulse having a constant voltage level for the duration of the positive phase, the cell membrane response to the positive phase reaches a peak, i.e., at an optimum level, at the trailing edge of the positive phase. However, voltage delivered via a charged capacitor does not necessarily remain at a constant voltage level, but rather may have some degree of a “tilt” or a discharge slope associated with it, i.e., the percent drop in voltage from the beginning to the end of each phase. Such tilt may cause the peak cell membrane response to occur at some point prior to the trailing edge of the positive phase. It is noted that exemplary antiarrhythmic therapies, according to the present disclosure, may have a tilt from about 70% to about 100%.

An exemplary treatment system may include an implantable therapy generator configured to generate and selectively deliver an atrial/ventricular antiarrhythmic therapy, and at least one lead, e.g., one, two, three lead(s), etc., operably connected to the implantable therapy generator, each lead having at least one electrode and/or at least one coil. Some exemplary systems may also be configured to administer cardiac resynchronization therapy (CRT). Thus, such exemplary systems may be configured to deliver the antiarrhythmic therapy discussed above in some instances, and CRT in other instances. Other exemplary systems may be configured to deliver the antiarrhythmic therapy and CRT simultaneously.

The therapy generator may include a battery system operably coupled and providing power to sensing circuitry, detection circuitry, control circuitry, and therapy circuitry of the implantable therapy generator. The sensing circuitry may sense cardiac signals representative of atrial activity and/or ventricular activity. The detection circuitry may evaluate the cardiac signals representative of atrial/ventricular activity to determine an atrial/ventricular cycle length and detect an atrial/ventricular arrhythmia based at least in part on the atrial/ventricular cycle length, or variation in the atrial/ventricular cycle length. The control circuitry, in response to the atrial/ventricular arrhythmia, may control generation and selective delivery of a single-stage or a multi-stage antiarrhythmic therapy to the electrodes. The multi-stage therapy may have inter-stage delay(s) during the antiarrhythmic therapy. The therapy circuitry may be operably connected to the electrodes and the control circuitry, and may, for example, include at least one first stage charge storage circuit selectively coupled to at least one far field electrode that selectively stores energy for a first stage of a single-stage or multi-stage antiarrhythmic therapy. In other examples, the therapy circuitry may further include at least one second stage charge storage circuit selectively coupled to at least one far field electrode that selectively stores energy of a second stage of a multi-stage antiarrhythmic therapy, and/or at least one third stage charge storage circuit selectively coupled to a near field electrode that selectively stores energy of a third stage of a multi-stage antiarrhythmic therapy. It is noted that, in some other embodiments, upon detection of arrhythmia via the detection circuitry, the control circuitry may be configured or programmed to deliver an order of therapies. For example, the order may include a first therapy of ATP, and a second therapy of a single-stage or multi-stage antiarrhythmic therapy according to this disclosure.

It is noted that the positioning of each of the leads may be dependent on whether the system is configured to deliver an atrial antiarrhythmic therapy or a ventricular antiarrhythmic therapy, both of which are further described below. For example, to optimize the above-described therapy, multiple electric field configurations may be used to optimally induce virtual electrodes that can depolarize (excite) the excitable gap near the core of reentry and disrupt the reentrant circuit. These field configurations may be achieved by placing defibrillation/CRT leads and electrodes into the right atrium, right ventricle, coronary sinus, left atrium, and/or the left ventricular veins, depending on the type of therapy. The leads may be of active or passive fixation. Electric fields may be delivered between any two or more of these electrodes as well as between one of these electrodes and the generator itself (i.e., a hot can configuration). Modulation of the electric field vector may be used to achieve maximum coverage of the portion of the heart to be treated. The system may also be programmed with a set of therapy parameters for delivering the antiarrhythmic therapy to a patient via a far-field configuration and/or a near-field configuration of the electrodes upon detection of an arrhythmia by the system.

It is noted that above discussed treatment systems, or at least a portion thereof, are not limited to transvenous systems, but may also be subcutaneous or epicardial systems. For example, the above discussed treatment systems may be implanted subcutaneously so that the leads and electrodes are not in direct contact with the heart, or may be placed epicardially, on the outside of the heart. The placement of a subcutaneous or epicardial treatment system is not particularly limited.

For example, a subcutaneous treatment system may be positioned around any portion of the torso, or other body locations, that may be suitable for the delivery of atrial antiarrhythmic therapy or ventricular antiarrhythmic therapy according to aspects of this disclosure. It is also understood that different aspects of the treatment system may be subcutaneously located at several different body locations, such as in the chest, abdominal, or subclavian region with leads and electrodes respectively positioned around different regions of the heart. For example, a therapy generator of a subcutaneous system may be configured for positioning outside of a rib cage at an intercostal or subcostal location, within the abdomen, or in the upper chest region (e.g., subclavian location). In some embodiments, the therapy generator may be an “active” can. Stated differently, a portion of the housing of the therapy generator may be an electrode configured for delivering therapy. One or more leads and electrodes may be located on the generator and/or at other locations about, but not in direct contact with, the heart or cardiac vessels. Thus, exemplary atrial/ventricular antiarrhythmic therapies, according to aspects of this disclosure, may also be deliverable via subcutaneous treatment systems.

Epicardial treatment systems may be positioned so at least the leads and electrodes of the system may be epicardially positioned on the heart. The manner by which the leads and electrodes are positioned onto the heart is not particularly limited, and may be by, for example, suturing, adhesion, etc. The position of the therapy generator is not particularly limited, and may be any suitable position that may securely hold the therapy generator in place, e.g., the torso. Thus, it is understood that different aspects of the exemplary treatment system may be located at different body locations, such as the therapy generator being around the torso region and the leads and electrodes respectively positioned on the heart. For example, a therapy generator of an epicardial system may be positioned in a pocket formed by a surgeon that is between layers of the upper chest wall or the abdomen muscles. Thus, exemplary atrial/ventricular antiarrhythmic therapies, according to aspects of this disclosure, may also be deliverable via epicardial treatment systems.

Other exemplary treatment systems may be leadless and/or battery-free. Thus, such treatment systems may be externally controlled and/or programmable. An exemplary leadless system may comprise an external transmitter in communication with an implantable leadless treatment device. The external transmitter may be adapted for the wireless delivery of power to the device and/or control of the treatment device. The means by which the delivery ensues is not particularly limited, and in some examples, may be via wireless, resonant inductive coupling between transmitting features of the external transmitter and the treatment device. Thus, the external transmitter may help eliminate the need for batteries and may also allow for externalized control without transcutaneous leads. Similar to the above-discussed implantable therapy generator, the external transmitter may comprise sensing circuitry, detection circuitry, control circuitry, and therapy circuitry. Thus, the external transmitter, in conjunction with the treatment device, may be configured for the identification of atrial/ventricular arrhythmia and the delivery of an atrial/ventricular antiarrhythmic therapy according to aspects of this disclosure.

The implantable treatment device may comprise at least a power source, a receiver adapted for receiving wireless transmissions from the external transmitter, and at least one electrode, e.g., one, two, three, four, etc. electrodes, configured to be in contact with the heart. The power source is not particularly limited, and may be any suitable power source that may act as a power harvester in communication with the external transmitter. For example, the power source may be an external battery, a rechargeable, implantable capacitor, etc. configured for inductive power transfer with the external transmitter. Likewise, the receiver is not particularly limited, and may be any suitable receiving source that may receive wireless transmissions from the external transmitter. The at least one electrode may be any suitable electrode (or electrodes) configured to deliver far field pulses and near field pulses, in accordance with therapies contemplated throughout this disclosure.

The treatment device may further include a casing at least partially covering the aforementioned power source, receiver, and at least one electrode. The receiver, at least one electrode, and casing may be of any suitable flexible material. Thus, the treatment device may flex and mold to the outer shape of a heart. The treatment device may be in direct contact with any suitable portion of the heart, depending on the treatment (atrial or ventricular antiarrhythmic therapy) to be administered. The manner by which the treatment device is in contact with the heart is not particularly limited, and may be, for example, via adhesion, suture, etc.

Furthermore, some exemplary implantable leadless devices may also be fully bioresorbable. Such device may comprise of any suitable materials that resorb when exposed to biofluids in a time-controlled manner via metabolic action and hydrolysis, e.g., tungsten-coated magnesium (W/mg), poly(lactide-co-glycolide) (PLGA), silicon nanomembrane (Si NM), etc. Thus, the device may disappear completely through natural chemical/biological processes over a subsequent timeframe, thereby eliminating any need for surgical extraction of an implanted device. Such a timeframe for the operation of the device and complete bioresorption may be tailored to specific therapeutic timelines. In some examples, complete bioresorption may take place within five to seven weeks, but is not limited thereto.

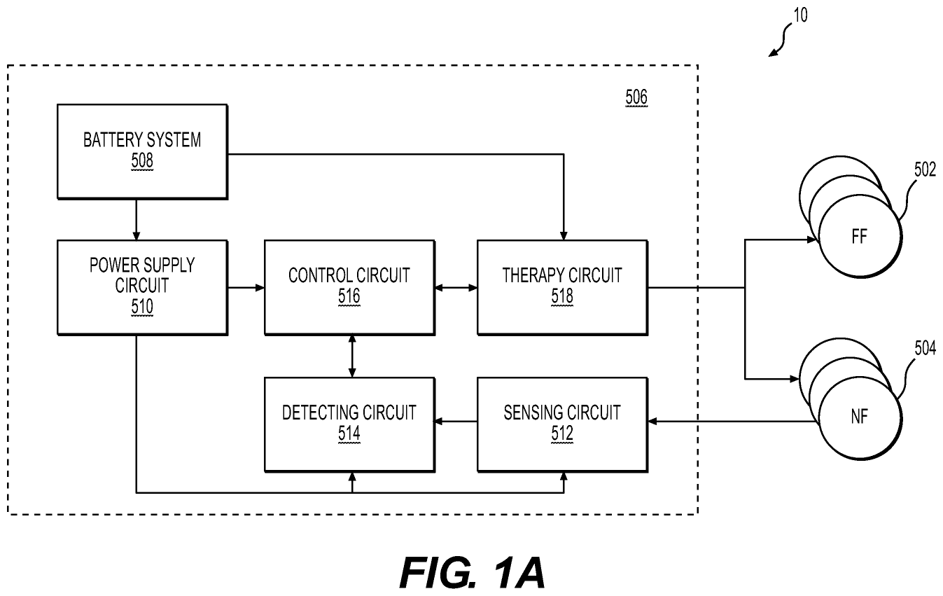

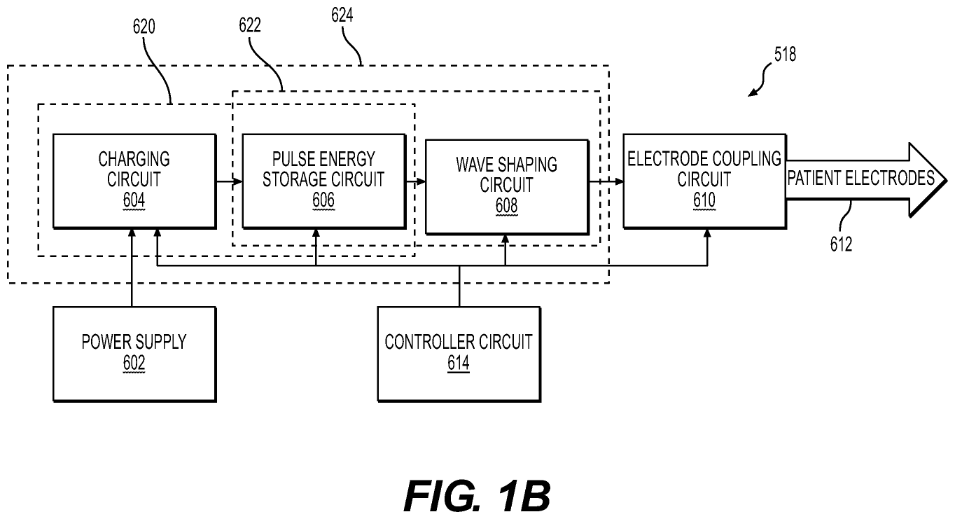

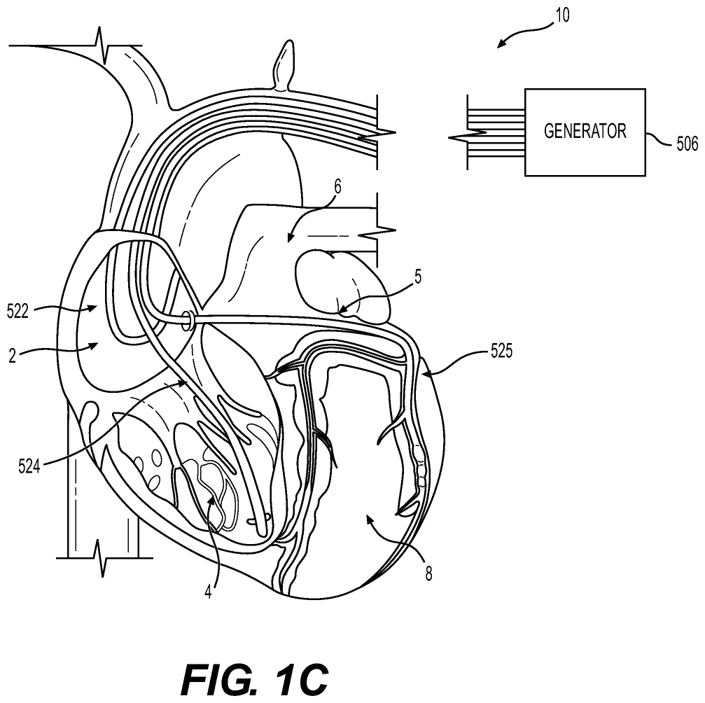

Referring now to FIGS. 1A and 1B, an exemplary antiarrhythmic therapy system 10 is described. System 10 includes a generator 506, a plurality of leads (shown in FIG. 1C), a plurality of electrodes 502 adapted to be implanted adjacent to an atrium/ventricle of a heart of a patient to deliver far field pulses, and a plurality of electrodes 504 adapted to implanted proximate the atrium/ventricle of the heart of the patient to deliver near field pulses and sense cardiac signals. The housing of generator 506 may serve as one of the far-field electrodes 502 or near-field electrodes 504. Additionally, in some embodiments, far-field electrodes 502 and near-field electrodes 504 may share at least one common electrode.

Generator 506 may be operably connected to electrodes 502, 504, and may include a battery system 508 (or other suitable on-board energy sources, e.g., super capacitors) and one or more power supply circuits 510 operably coupled and providing power to sensing circuitry 512, detection circuitry 514, control circuitry 516, and therapy circuitry 518 of generator 506. In some embodiments, therapy circuitry 518 may include a specialized power supply that is fed directly from battery system 508, bypassing power supply circuitry 510. Sensing circuitry 512 may sense cardiac signals representative of atrial activity and/or ventricular activity. Detection circuitry 514 may evaluate cardiac signals representative of atrial/ventricular activity to determine an atrial/ventricular cycle length and detect an atrial/ventricular arrhythmia based at least in part on the atrial/ventricular cycle length. Control circuitry 516, in response to the arrhythmia, may control generation, and selective delivery of the atrial/ventricular antiarrhythmic therapy to electrodes 502 and 504. In various embodiments, detection circuitry 514, control circuitry 516 and therapy circuitry 518 may share components. For example, in an embodiment, a common microcontroller may be a part of detection circuitry 514, control circuitry 516, and therapy circuitry 518.

Therapy circuitry 518 may be operably connected to electrodes 502 and 504 and control circuitry 516. FIG. 1B illustrates an exemplary arrangement of therapy circuitry 518. Therapy circuitry 518 may include its own power supply circuit 602, which may be fed from battery system 508. Power supply circuit 602 may be a simple voltage regulator, or it may be a current limiting circuit that functions to prevent therapy circuitry 518 from drawing too much power and, consequently, causing a drop in the supply voltage below a sufficient level to power the controller and other critical components. Alternatively, power supply circuit 602 may be implemented in power supply circuit 510, or, in another embodiment, power supply circuit 602 can be omitted entirely, such that charging circuit 604 is fed directly from battery system 508.

With reference now to FIG. 1B, charging circuit 604 may be a voltage converter circuit that produces voltages at the levels needed for the stimulation waveform. Since the stimulation waveform, particularly the first stage, may be at a much higher voltage relative to other stages of the therapy, a boosting topology may be used for charging circuit 604. It is noted that the voltage of the first stage may still be a fraction of a typical voltage used for a standard defibrillation shock, e.g., a first stage voltage less than 230 V. Any suitable boosting circuit may be employed to this end, including a switching regulator utilizing one or more inductive elements (e.g., transformer, inductor, etc.), or a switching regulator utilizing capacitive elements (e.g., charge pump). Pulse energy storage circuit 606 may take various forms, and is not particularly limited. Generally, pulse energy storage circuit 606 may have energy storage capacity sufficient to store either all possible stages of the atrial/ventricular therapy, or a portion of the therapy's energy, provided that the arrangement of energy storage circuit 606 and charging circuit 604 supports the ability to re-charge portions of the energy storage circuit 606 while other portions thereof are discharging or are about to discharge during application of the electrotherapy. Wave shaping circuit 608 may be any suitable circuit adapted for regulating the application of the electrotherapy by selecting, and controlling the discharging of the energy stored in energy storage circuit 606.

FIG. 1C illustrates an anterior view of a heart and system 10 at least partially implanted within the heart. Notably, FIG. 1C illustrates an exemplary lead configuration of system 10. System 10 includes a first lead 522 within right atrium 2, a second lead 524 within right ventricle 4, and a third lead 525 extending within coronary sinus 5 so that lead 525 is proximate both left atrium 6 and the left ventricle 8. As discussed above, each of first lead 522 and second lead 524 may include at least one electrode and/or at least one defibrillation coil, and third lead 525 may include at least one electrode, as shown in other embodiments discussed further below. Moreover, in other exemplary embodiments, system 10 may be without, for example, first lead 522 or second lead 524, depending on the type of therapy system 10 may be adapted for. System 10 (and other exemplary devices) may be implanted just under a left clavicle or a right clavicle of a patient, depending on the presence of other devices, anatomical considerations, and/or patient lifestyle factors. This location may place system 10 in approximate alignment with the longitudinal anatomical axis of the heart (an axis through the center of the heart that intersects the apex and the inter-ventricular septum).

It is noted that lead positioning may be assessed via a plurality of post-surgical views. For example, such views include coronary angiography standard views, which include a right anterior oblique (RAO) view (in which an imager is rotated to a patient's right so that the spine is on the right side of the image) and a left anterior oblique (LAO) view (in which an imager is rotated to a patient's left so that the spine is on the left side of the image). Two criteria for assessing optimal lead position via the RAO and LAO fluoroscopic views are: 1) contact with the atrial septal wall; and 2) coverage of the left atrium between first lead 522 in the right atrium and third lead 525 in the coronary sinus. Septal wall contact may be assessed via motion of a lead or electrode relative to the septal wall. Coverage of the left atrium may be assessed by the relative position of first lead 522, third lead 525, and/or catheters in relation to one other and first lead 522 and third lead 525 may appear parallel relative to one another in the fluoroscopic views. Therapy is most efficacious when the induced virtual electrodes are proximal to the wavefronts and can destabilize or terminate the reentrant circuit or wavefronts that are maintaining AF.

System 10 may be fully automatic, automatically delivering a shock protocol when atrial/ventricular arrhythmias are detected. In other examples, system 10 may have a manual shock delivery so that system 10 prompts the patient, via any suitable means, to either have a doctor authorize generator 506 to deliver a shock protocol, or system 10 can prompt the patient to self-direct generator 506 to deliver a shock protocol in order to terminate a detected arrhythmia. In some other examples, system 10 may be semi-automatic; a “bed-side” monitoring station may be used to permit remote device authorization for the initiation of a shock protocol when arrhythmias are detected.

Further discussion of atrial and ventricular antiarrhythmic therapies, as well as systems adapted for such therapies, is provided below.

Atrial Antiarrhythmic Therapy

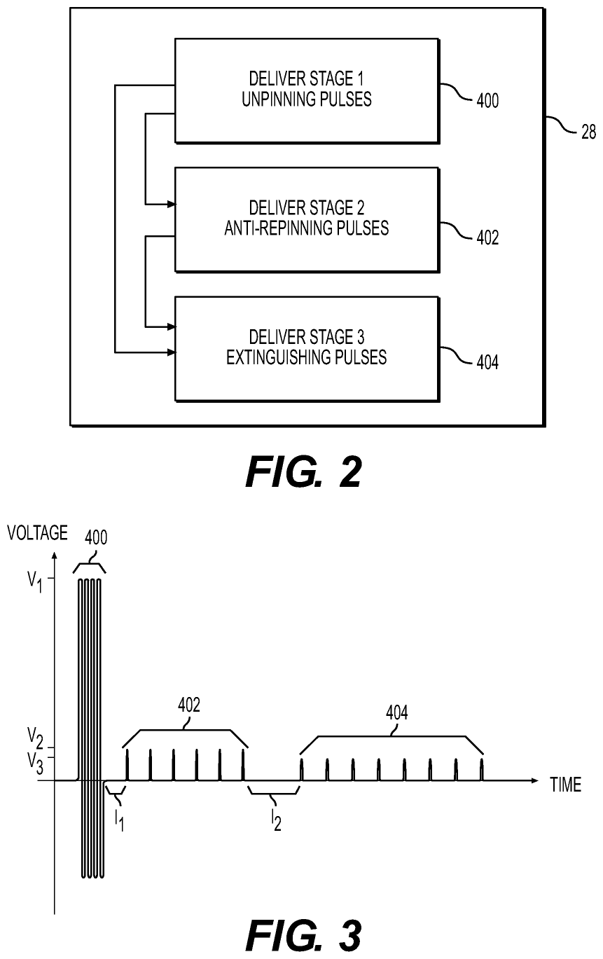

FIG. 2 illustrates an exemplary atrial antiarrhythmic therapy 28 that may be delivered to a patient in response to a detection of an atrial arrhythmia, e.g., AF or AFl. As discussed above, therapy 28 may be a single-stage therapy including only a first stage 400, or may be a multi-stage therapy including first stage 400, and one or both of a second stage 402 and a third stage 404. The various combinations defining therapy 28 may be indicated by the directional arrows shown in FIG. 2. The delivery of first stage 400, second stage 402, and third stage 404 may be via various configurations of electrodes. For example, first stage 400 and second stage 402 may be delivered via a far field configuration of electrodes, whereas third stage 404 may be delivered via a near field configuration of electrodes in one embodiment of therapy 28. Each of stages 400, 402, 404 may have its respective set of therapy parameters.

It is noted that, in some examples, therapy 28 may include multiple single-stage or multi-stage therapies. In such examples, a single-stage or multi-stage therapy may be followed by another therapy, which may also be single-stage or multi-stage depending on the assessment of the atrial arrhythmia to be treated. Such an assessment may be made subsequently between stages or between therapies. It is further noted that, in some other multi-stage therapy examples, transition between stages may take place without any assessment in between the therapy stages. Thus, it may be understood that “multi-therapy” includes multiple atrial antiarrhythmic therapies.

Referring to FIG. 3, an exemplary combined representation of all three of the stages of an atrial antiarrhythmic therapy is shown. Again, those of ordinary skill in the art will recognize that any of the depicted stages may be delivered without the other stages, or in combination with any or all of the other depicted stages. First stage 400 is applied for unpinning and/or extinguishing one or more singularities associated with an atrial arrhythmia. Second stage 402 may be applied for anti-repinning and/or extinguishing the one or more singularities associated with the atrial arrhythmia. Third stage 404 may be applied for extinguishing of the one or more singularities associated with the atrial arrhythmia. As shown, a first interstage delay I₁ may be between first stage 400 and second stage 402, and a second interstage delay 12 may be between second stage 402 and third stage 404. It is noted that, in some examples, there may be no further sensing or assessments during the interstage delays I₁ and I₂. Alternatively, in other examples, further sensing and/or assessments may take place between interstage delays I₁ and I₂. Furthermore, as shown, the voltage and duration (among other therapy parameters) of each of the respective stages 400, 402, 404 may be different from one other as well.

Because the delivery of electrical stimuli to the atria beyond a certain period of time may induce ventricular arrhythmia, the duration of atrial antiarrhythmic therapy 28 may be defined so that such induction is avoided. In a standard electrocardiogram (EKG), the T-wave occurs when the ventricles repolarize and the greatest dispersion of refractoriness is present. This dispersion of refractoriness present during the T-wave results in the ventricles becoming vulnerable to electrical stimuli, which may induce an arrhythmia. As a result, the T-wave may be referred to as the vulnerable period. Thus, in view of this vulnerable period, a number of precautions may be taken. For example, the time-duration of first stage 400 may be defined so that it is not longer than the S-T segment of the EKG. Moreover, the voltage of second stage 402 may be set to less than or equal to approximately 60% of the Ventricular Shock Excitation Threshold (VSET) to ensure that therapy delivered to the atria does not affect the ventricle, as second stage 402 will occur during the T-wave. VSET may be defined as the minimum energy by which a monophasic 10-ms shock excites the ventricle. Because second stage 402 would occur during the T-Wave, a measurement of VSET may be taken to ensure that second stage 402 does not elicit a response in the ventricles and risk inducing ventricular arrhythmia. VSET may be measured by progressively increasing the strength of a far field monophasic 10 ms pulse and determining at what strength the ventricle is captured via EKG. Both of the above precautions taken in first stage 400 and second stage 402 may minimize the risk of inducing a ventricular arrhythmia during therapy 28.

In various embodiments, first stage 400 may have at least two pulses, e.g., three, four, five, six, seven, eight, nine, ten, etc., of approximately 10 volts to approximately 150 volts, e.g., approximately 100 volts. An energy of each pulse is not particularly limited, and may be, for example, at least 0.01 joules. Each pulse may be less than or equal to approximately 20 ms, and in some instances, may be less than or equal to approximately 15 ms or approximately 10 ms. Each pulse of first stage 400 may be a biphasic waveform. For example, a biphasic pulse of approximately 10 ms may be approximately 6 ms of a first phase and approximately 4 ms of a second phase. Moreover, a voltage of the second phase may be a function of the voltage of the first phase of the biphasic pulse, e.g., approximately 30% to approximately 70%, or preferably approximately 50%, of the first phase. For example, a leading edge voltage of the first phase may have a voltage of approximately 100 volts and a leading edge voltage of the second phase may have a voltage of approximately 50 volts. However, first stage 400 pulses are not limited thereto, and may alternatively comprise monophasic or other custom-configured pulses in some other examples. Each pulse of first stage 400 may be delivered approximately 20 to approximately 50 ms apart, e.g., approximately 25, 30, 35, 40, or 45 ms apart. As noted above, in some embodiments, first stage 400 may have a total duration that is less than the S-T segment, e.g., approximately 140 ms to approximately 180 ms, of a standard electrocardiogram (EKG). First interstage delay I₁ of approximately 50 to approximately 400 milliseconds may precede second stage 402, though in other embodiments, first interstage delay I₁ may be of a longer or shorter duration, e.g., approximately 55 ms to approximately 300 ms.

In various embodiments, second stage 402 may have at least four pulses, e.g., six, eight, ten, etc., of less than or equal to 60% of VSET, e.g., approximately 50-60% of VSET. This may correspond to less than or equal to approximately 20V. Each pulse of second stage 402 may be a monophasic waveform, but not limited thereto. Each pulse may be less than or equal to approximately 20 ms, and in some instances, may be less than or equal to approximately 15 ms or approximately 10 ms. Each pulse of second stage 402 may be delivered approximately 10 ms to approximately 300 ms apart, and in some examples, this pulse coupling interval may be defined as approximately 70-100% of an atrial arrhythmia cycle length, e.g., approximately 88% of atrial arrhythmia cycle length. The duration of second stage 402 may be between approximately 400 ms to approximately 3500 ms. Second interstage delay I₂ of approximately 55 ms to approximately 300 milliseconds may precede third stage 404.

In various embodiments, third stage 404 may have at least five pulses, e.g., six, seven, eight, nine, ten, etc., of less than or equal to approximately 20 volts. Each pulse of third stage 404 may be a monophasic waveform, but not limited thereto. Each pulse may have a pulse duration of between approximately 0.1 ms to approximately 5 ms. Each pulse of third stage 404 may be delivered approximately 10 ms to approximately 300 ms apart, and in some examples, this pulse coupling interval may be defined as approximately 70-100% of an atrial arrhythmia cycle length, e.g., approximately 88% of atrial arrhythmia cycle length. Thus, the pulses of third stage 404 may be pacing-like pulses. The duration of third stage 404 may be between approximately 500 ms to approximately 4000 ms.

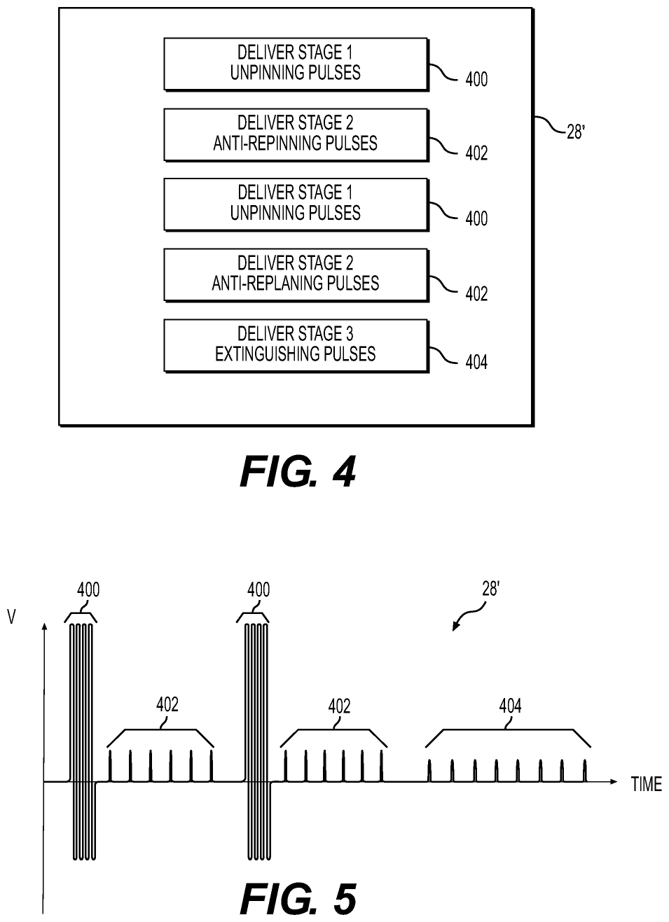

Referring to FIGS. 4 and 5, a multi-therapy embodiment of the atrial antiarrhythmic therapy 28′ is shown. In this embodiment, first stage 400 and second stage 402 may each be repeated in sequence as part of the overall atrial antiarrhythmic multi-therapy 28′, before delivery of third stage 404. The therapy parameters for each of stages 400, 402, 404, and each of the pulses within each stage, may be the same or different for different stages and/or different pulses within each stage.

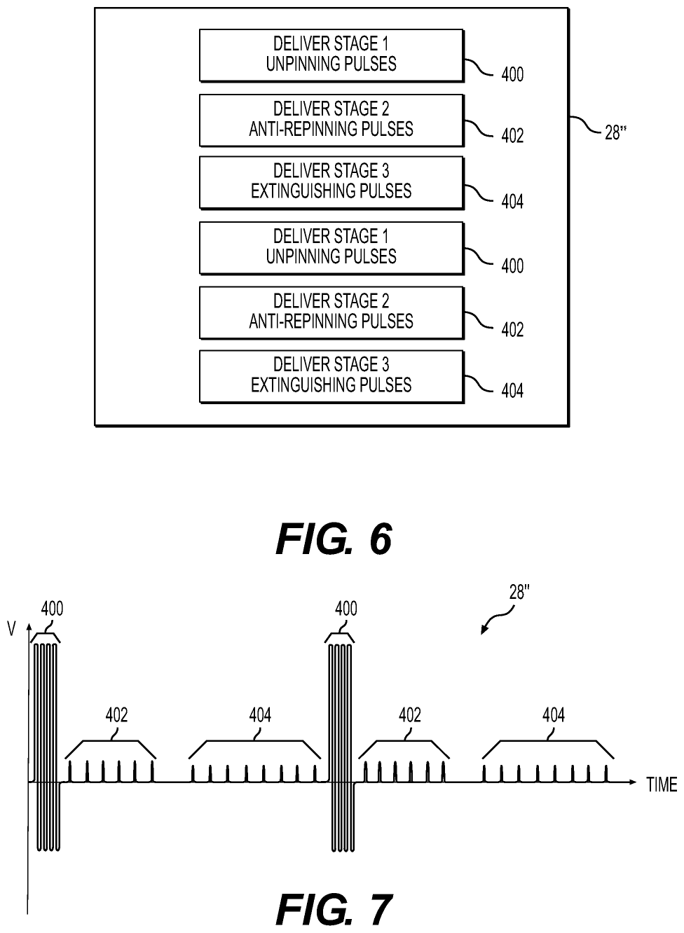

Referring to FIGS. 6 and 7, another multi-therapy embodiment of the atrial antiarrhythmic therapy 28″ is shown. In this embodiment, first stage 400 and second stage 402, as well as third stage 404 are each repeated in as part of the overall atrial antiarrhythmic multi-therapy 28″, followed by a repeated delivery of all three of the stages before completion of atrial antiarrhythmic therapy 28″. The therapy parameters for each of stages 400, 402, 404, and each of the pulses within each stage, may be the same or different for different stages and/or different pulses within each stage.

Atrial Antiarrhythmic Therapy Systems

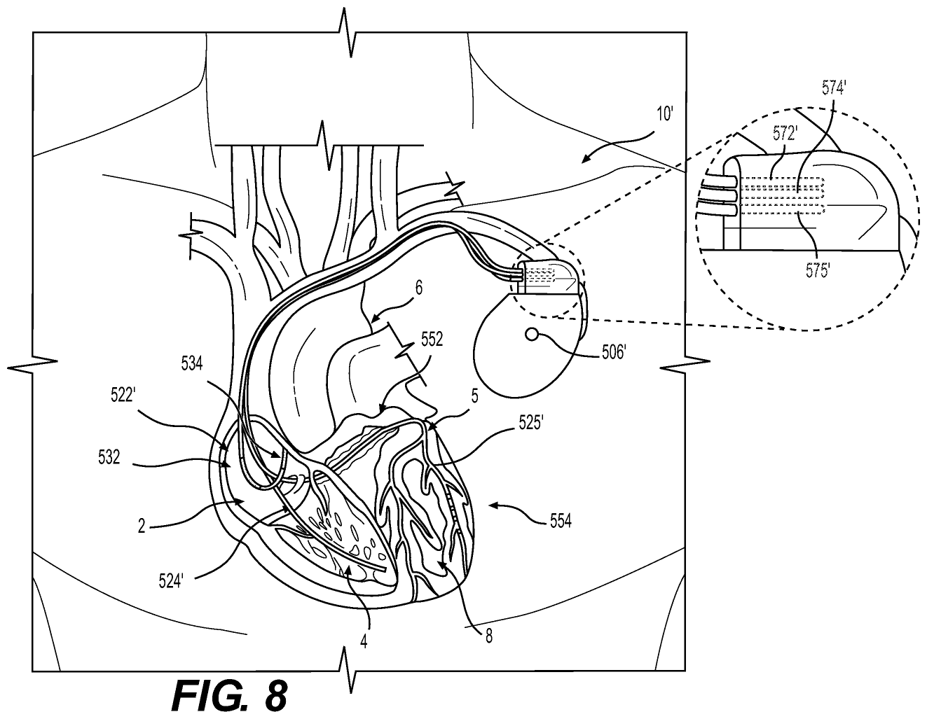

FIG. 8 illustrates an anterior view of a heart and an exemplary system 10′ at least partially implanted within the heart. System 10′ is similar to system 10, as shown in FIG. 1C, in some respects, and like reference numerals refer to like parts. System 10′ includes a generator 506′, a first lead 522′ within right atrium 2, a second lead 524′ within right ventricle 4, and a third lead 525′ extending within coronary sinus 5 so that lead 525′ is proximate both left atrium 6 and the left ventricle 8. However, in some exemplary embodiments, system 10′ may be without second lead 524′, and, in other exemplary embodiments, may only include a single pass lead.

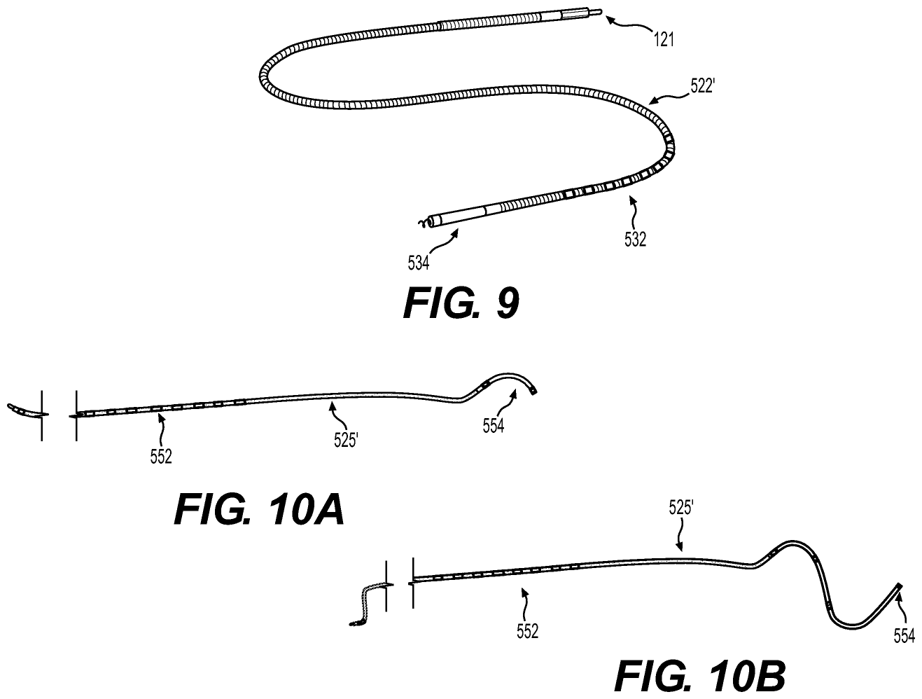

First lead 522′ includes a first end coupled to generator 506 and a second end proximate right atrium 2. As shown in FIG. 9, the first end may include a connective feature 121 that may be received by a feature of generator 506, e.g., a connective header. First lead 522′ further includes at least one pacing electrode 534 at the second end and at least one therapy electrode 532 along a portion of lead 522′ between the first and second ends. Therapy electrodes 532 may be configured for far-field and/or near-field stimulation. Thus, therapy electrodes 532 may deliver a first stage, second stage, and/or a third stage of an atrial antiarrhythmic therapy. The number of therapy electrodes 532 and pacing electrodes 534 is not particularly limited. As shown in FIG. 9, the number of therapy electrodes 532 may be, for example, eight electrodes, but not limited thereto. It is noted that in some embodiments pacing electrode 534 may be in a tip-ring form. Moreover, in some embodiments, therapy electrode 532 may be substituted with a defibrillation coil. Therapy electrode 532 may be positioned so that it may be proximate, or adjacent to, the atrial septum. In some embodiments, lead 522′ may be biased towards a septal wall so that therapy electrode 532 may be positioned accordingly.

Second lead 524′ is not particularly limited, and includes a first end coupled to generator 506 and a second end proximate right ventricle 4. Second lead 524′ may be dormant during the delivery of an atrial antiarrhythmic therapy, and as noted above, atrial antiarrhythmic therapy system 10′ may be without second lead 524′ in some embodiments. In another embodiment, second lead 524′ may include at least one pacing electrode on the distal portion of the lead and two sets of far-field electrodes. Between the two sets of far-field electrodes, the distal set of electrodes may be located proximate the right ventricle and the proximal set of electrodes may be located proximate the right atrium, thereby allowing for far-field stimulation via the proximal set of electrodes and third lead 525′. The spacing between the two sets of electrodes may allow for the proximal set of electrodes to be proximate the right atrium.

Third lead 525′ includes a first end coupled to generator 506 and a portion that is proximate left atrium 6 and left ventricle 8. Third lead 525′ further includes at least one pacing electrode 554 proximate left ventricle 8 and at least one therapy electrode 552 along the portion of lead 525′ proximate left atrium 6. Therapy electrodes 552 may be configured for far-field and/or near-field stimulation. Thus, therapy electrodes 552 may deliver a first stage, second stage, and/or a third stage of an atrial antiarrhythmic therapy. The number of therapy electrodes 552 and pacing electrodes 554 is not particularly limited. In some embodiments, lead 525′ may include a plurality of pacing electrodes 554, e.g., bipole or tripole as shown in FIGS. 10A and 10B respectively, and may be configured for CRT delivery. Likewise, in some embodiments, lead 525′ may include a plurality of therapy electrodes 552, e.g., eight electrodes as shown in FIGS. 10A and 10B, (or a defibrillation coil) that may be positioned within the CS on a lateral side or the posterior wall of left atrium 6. A bias may be created in the distal or mid portion of lead 525′ in order to aid fixation and stability.

Generator 506′ is not particularly limited and may include a plurality of connectors or headers 572′, 574′, 575′, each of which may be coupled to the first end of a respective lead 522′, 524′, 525′. Each of headers 572′, 574′, 575′ may be configured to deliver a sufficient amount of power to its respective lead for atrial antiarrhythmic therapy. For example, header 572′, which may be coupled to a first end of lead 522′, may be an IS-4 (LLLL) or a DF-4 (LLHH) header, which may deliver more power relative to a standard IS-1 header. In another example, header 575′, which may be coupled to a first end of lead 525′, may be a DF-4 (LLHH) header, the H being equal to or above 20 V, thereby providing a sufficient degree of power to a left atrium and ventricle of the heart. It is noted that generator 506′ may be without header 574′ in embodiments which lead 524′ is not present within system 10′.

In other exemplary embodiments, in which lead 524′ is used with a single set of far-field electrodes, a Y-adapter connected to port 574′, which may be a DF-4 (LLHH) header, may be used to divert a H from the DF-4 (LLHH) header to supply power that is greater than 20V to a set of far-field electrodes on lead 525′. In this embodiment, lead 525′ would consist of quadripolar pacing electrodes at the distal end with at least one set of far-field electrodes that would connect to the Y-adapter from the header via a bifurcation on the proximal end of lead 525′ to yield five circuits (IS-4, LLLL for the near-field electrodes, and a DF-1 for the far-field electrodes.)

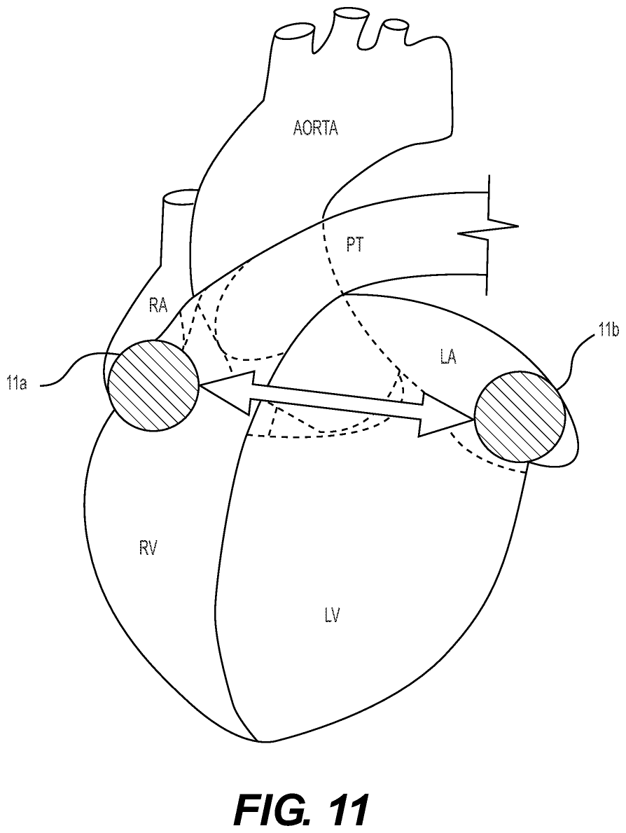

As shown in FIG. 11, in accordance with atrial antiarrhythmic system 10′, at least one therapy electrode 532 configured for far-field/near-field stimulation may be positioned in a region 11a, which may be proximate the right atrium. Moreover, at least one therapy electrode 552 configured for far-field/near-field stimulation may be positioned in a region 11b, which may be proximate the left atrium. Thus, an electric field of the atrial antiarrhythmic therapy may be delivered between regions 11a and 11b, as indicated by the directional arrow shown FIG. 11.

It is noted that electric fields may be delivered, in any direction, between any two therapy electrodes, e.g., electrodes 532 and 552, as well as between a therapy electrode and generator 506′ itself (hot can configuration). Thus, the vector/polarity of the stage(s) is not particularly limited. Modulation of the electric field vector/polarity may be used to achieve maximum coverage of the entire atria and to maintain optimal Virtual Electrode Polarization pattern in order to depolarize the maximum area of excitable gaps. The optimal electric fields used and the correct sequence of fields may also be explored on a trial and error basis for each patient or may be estimated based on external information regarding potential sites of the reentrant circuits, or may be based on a combination of both.

Ventricular Antiarrhythmic Therapy

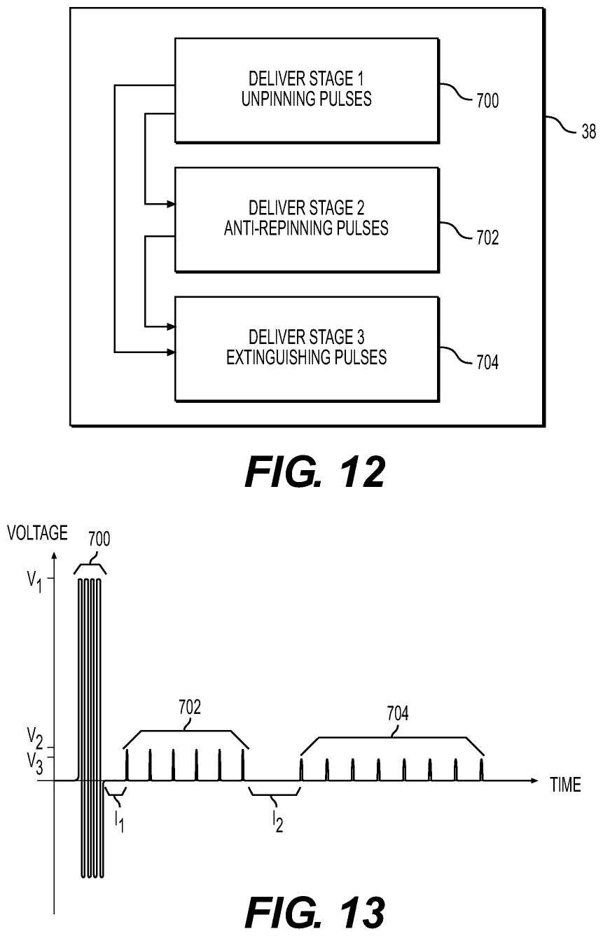

FIG. 12 illustrates an exemplary ventricular antiarrhythmic therapy 38 that may be delivered to a patient in response to a detection of a ventricular arrhythmia, e.g., VF or VT. As discussed above, therapy 38 may be a single-stage therapy including only a first stage 700, or may be a multi-stage therapy including first stage 700, and one or both of a second stage 702 and a third stage 704. The various combinations defining therapy 38 may be indicated by the directional arrows shown in FIG. 12. The delivery of first stage 700, second stage 702, and third stage 704 may be via various configurations of electrodes. For example, first stage 700 and second stage 702 may be delivered via a far field configuration of electrodes, whereas third stage 704 may be delivered via a near field configuration of electrodes in one embodiment of therapy 38. Each of stages 700, 702, 704 may have its respective set of therapy parameters.

It is noted that, in some examples, therapy 38 may include multiple single-stage or multi-stage therapies, similar to as discussed above for therapy 28. As previously discussed, a single-stage or multi-stage therapy may be followed by another therapy, which may also be single-stage or multi-stage depending on the assessment of the atrial arrhythmia to be treated. Such an assessment may be made subsequently between stages or between therapies. Thus, it may be understood that “multi-therapy” includes multiple ventricular antiarrhythmic therapies, and that the exemplary multi-therapies illustrated in FIGS. 4-7 may also be applicable as ventricular antiarrhythmic therapies.

Referring to FIG. 13, an exemplary combined representation of all three of the stages of a ventricular antiarrhythmic therapy is shown. First stage 700 is applied for unpinning and/or extinguishing one or more singularities associated with a ventricular arrhythmia. Second stage 702 may be applied for anti-repinning and/or extinguishing the one or more singularities associated with the ventricular arrhythmia. Third stage 704 may be applied for extinguishing of the one or more singularities associated with the ventricular arrhythmia. As shown, a first interstage delay I₁ may be between first stage 700 and second stage 702, and a second interstage delay I₂ may be between second stage 702 and third stage 704. Furthermore, as shown, the voltage and duration (among other therapy parameters) of each of the respective stages 700, 702, 704 may be different from one other as well.

In various embodiments, first stage 700 of ventricular antiarrhythmic therapy 38 may have at least two pulses, e.g., three, four, five, six, seven, eight, nine, ten, etc., of approximately 2 volts to approximately 100 volts, and in some examples, approximately 10 volts to approximately 40 volts. An energy of each pulse is not particularly limited, and may be, for example, at least 0.001 joules. Each pulse may be less than or equal to approximately 20 ms, and in some instances, may be less than or equal to approximately 10 ms or approximately 6 ms. Each pulse of first stage 700 may be a monophasic waveform. However, first stage 700 pulses are not limited thereto, and may alternatively comprise other custom-configured pulses in some other examples. Each pulse of first stage 700 may be delivered approximately 30 ms to approximately 110 ms apart. In some embodiments, first stage 700 may have a total duration that is less than or equal to approximately 325 ms. First interstage delay I₁ of approximately 50 ms to approximately 800 ms may precede second stage 402, though in other embodiments, first interstage delay I₁ may be of a longer or shorter duration, e.g., 70 ms to 300 ms.

In various embodiments, second stage 702 may have at least four pulses, e.g, five, six, eight, ten, etc., of approximately 0.5 volts to approximately 20 volts. Each pulse of second stage 402 may be a monophasic waveform, but not limited thereto. Each pulse may be less than or equal to approximately 20 ms, and in some instances, may be less than or equal to approximately 15 ms or approximately 10 ms. Each pulse of second stage 402 may be delivered approximately 100 ms to approximately 300 ms apart. The duration of second stage 402 may be between approximately 400 ms to approximately 3500 ms. Second interstage delay 12 of 55 ms to 300 ms may precede third stage 404.

In various embodiments, third stage 704 may have at least five pulses, e.g., six, seven, eight, nine, ten, etc., of less than or equal to approximately 10 volts. Each pulse of third stage 404 may be a monophasic waveform, but not limited thereto. Each pulse may have a pulse duration of between 0.1 ms to 5 ms. Each pulse of third stage 404 may be delivered approximately 100 ms to approximately 300 ms apart. Thus, the pulses of third stage 404 may be pacing-like pulses. The duration of third stage 404 may be between approximately 500 ms to approximately 4000 ms.

Ventricular Antiarrhythmic Therapy Systems

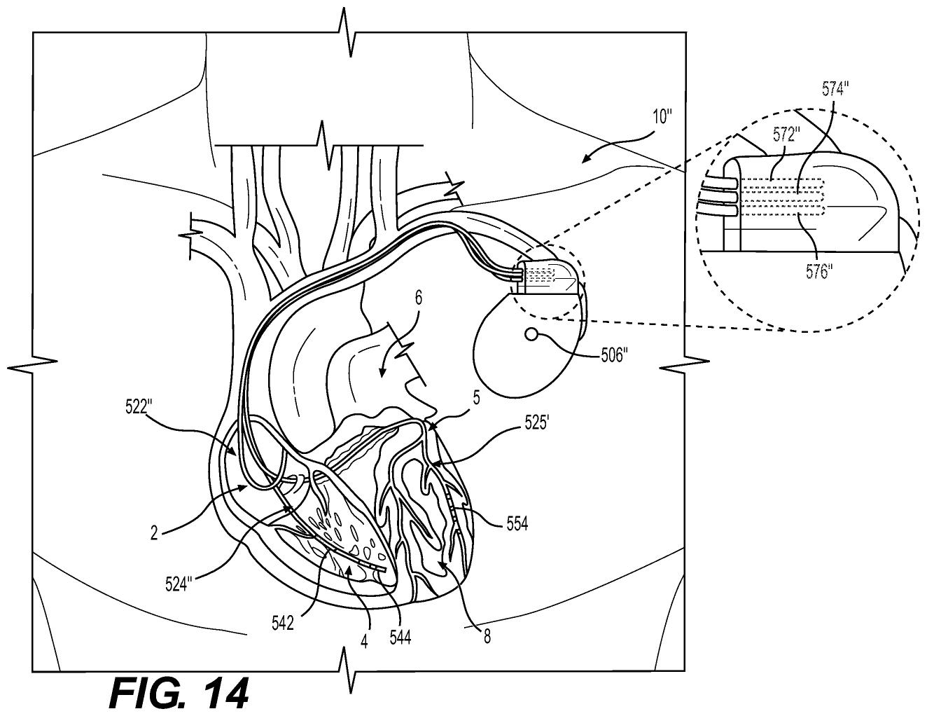

FIG. 14 illustrates an anterior view of a heart and an exemplary system 10″ at least partially implanted within the heart. System 10″ is similar to system 10′, as shown in FIG. 8, in some respects, and like reference numerals refer to like parts. System 10″ includes a generator 506″, a first lead 522″ within right atrium 2, a second lead 524″ within right ventricle 4, and a third lead 525″ extending within coronary sinus 5 so that lead 525″ is proximate both left atrium 6 and the left ventricle 8. However, in some exemplary embodiments, system 10″ may be without first lead 522″, and, in other exemplary embodiments, may include a single pass lead to replace first lead 522″ and third lead 525″.

First lead 522″ is not particularly limited, and includes a first end coupled to generator 506 and a second end proximate right atrium 2. First lead 522″ may be dormant during the delivery of a ventricular antiarrhythmic therapy, and as noted above, ventricular antiarrhythmic therapy system 10″ may be without first lead 522″ in some embodiments.

Second lead 524″ includes a first end coupled to generator 506″ and a second end proximate right ventricle 4. Second lead 524″ further includes at least one pacing electrode 544 at the second end and at least one therapy electrode 542 along a portion of lead 524″ that is proximate right ventricle 4. In some embodiments, therapy electrodes 542 may be configured for far-field stimulation and tip electrodes 544 may be configured for near-field stimulation. Thus, electrodes 542, 544 may deliver a first stage, second stage, and/or a third stage of a ventricular antiarrhythmic therapy. The number of therapy electrodes 542 and pacing electrodes 544 is not particularly limited. Moreover, in some embodiments, therapy electrode 542 may be substituted with a defibrillation coil.

Third lead 525″ includes a first end coupled to generator 506″ and a portion that is proximate left ventricle 8. Third lead 525″ further includes at least one pacing electrode 554 along the portion of lead 525″ that is proximate left ventricle 8. Pacing electrodes 554 may be configured for far-field and near-field stimulation. Thus, pacing electrodes 554 may deliver a first stage, second stage, and/or a third stage of a ventricular antiarrhythmic therapy. The number of pacing electrodes 554 is not particularly limited. In some embodiments, lead 525″ may include a plurality of pacing electrodes 554, e.g., a quadripole, and may be configured for CRT delivery.

Like generator 506′ discussed above, generator 506″ is not particularly limited and may include a plurality of connectors or headers 572″, 574″, 575″, each of which may be coupled to the first end of a respective lead 522″, 524″, 525″. Each of headers 572″, 574″, 575″ may be configured to deliver a sufficient amount of power to its respective lead for ventricular antiarrhythmic therapy. For example, header 574″, which may be coupled to a second end of lead 524″, may be an IS-4 header (LLLL). In another example, header 575″, which may be coupled to a first end of lead 525″, may be a DF-4 (LLHH) header, the H being less than or equal to approximately 20 V. It is noted that generator 506″ may be without header 572″ in embodiments which lead 522″ is not present within system 10″.

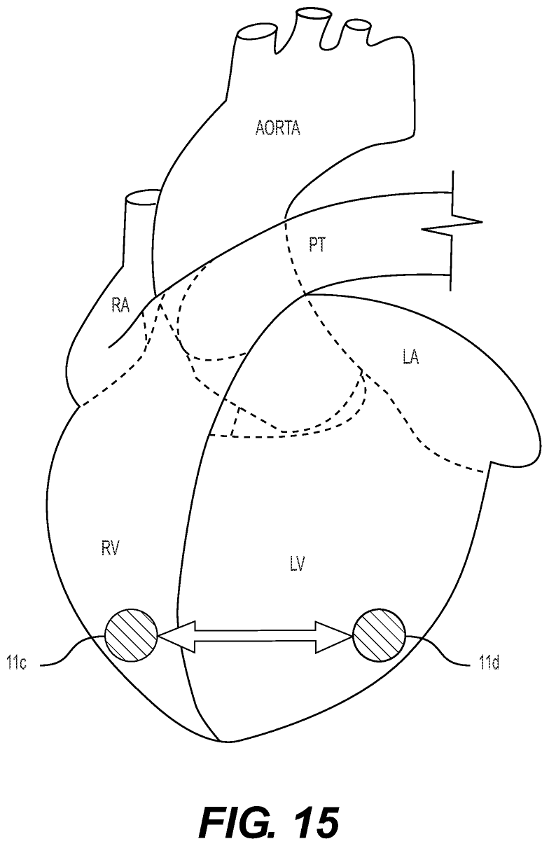

As shown in FIG. 15, in accordance with ventricular antiarrhythmic system 10″, electrodes 542, 544 configured for far-field/near-field stimulation may be positioned in a region 11c, which may be proximate the right ventricle. Moreover, at least one pacing electrode 554 configured for far-field/near-field stimulation may be positioned in a region 11d, which may be proximate the left ventricle. Thus, an electric field of the ventricular antiarrhythmic therapy may be delivered between regions 11c and 11d, as indicated by the directional arrow shown FIG. 15.

As previously discussed, electric fields may be delivered in any direction between any two therapy electrodes, e.g., electrodes 542 or 545 and 554, as well as between an electrode and generator 506″ itself in a hot can configuration. Thus, the vector/polarity of the stage(s) is not particularly limited. Modulation of the electric field vector/polarity may be used to achieve maximum coverage of the ventricles and to maintain optimal Virtual Electrode Polarization pattern in order to depolarize the maximum area of excitable gaps. The optimal electric fields used and the correct sequence of fields may also be explored on a trial and error basis for each patient or may be estimated based on external information regarding potential sites of the reentrant circuits, or may be based on a combination of both.

Additional Modalities

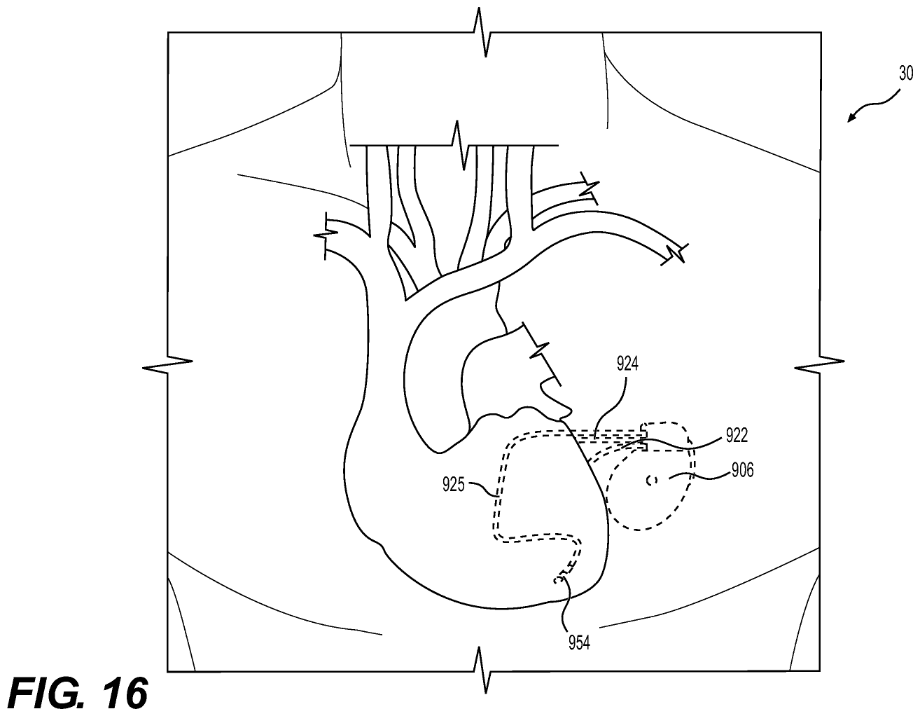

FIG. 16 illustrates another exemplary system 30 that is at least partially subcutaneously implanted and surrounding the heart. The subcutaneous positioning of system 30, relative to the heart, may be illustrated via the dashed lines depicting system 30. It is noted that system 30 may be similar to systems 10′ and 10″, apart from the at least partial subcutaneous implantation of system 30. Like systems 10′ and 10″, system 30 may comprise a generator 906, which may be similar to generator 506′, 506″, a plurality of leads 922, 924, 925, which may, respectively, be similar to leads 522′, 522″, 524′, 524″, 525′, 525″, and each of the aforementioned leads may include at least one electrode 954 (other electrodes not shown), which may be similar to any one of electrodes 532, 534, 542, 544, 552, 554.

As discussed above, the subcutaneous placement of system 30 is not particularly limited, and may, for example, be positioned around any portion of the torso, or other body locations, that may be suitable for the delivery of any one of the atrial antiarrhythmic therapies or ventricular antiarrhythmic therapies discussed or contemplated throughout this disclosure. For example, as shown in FIG. 16, generator 906 may be placed around a left mid-clavicular line, approximately at the level of upper or mid rib cage, and leads 922, 924, 925 may traverse in a subcutaneous path around various aspects of the heart. Each of leads 922, 924, 925 may traverse around the heart so that it may be adjacent to an atrium/ventricle of a heart of a patient to deliver far field and near field pulses, in accordance with the atrial/ventricular antiarrhythmic therapies according to this disclosure.

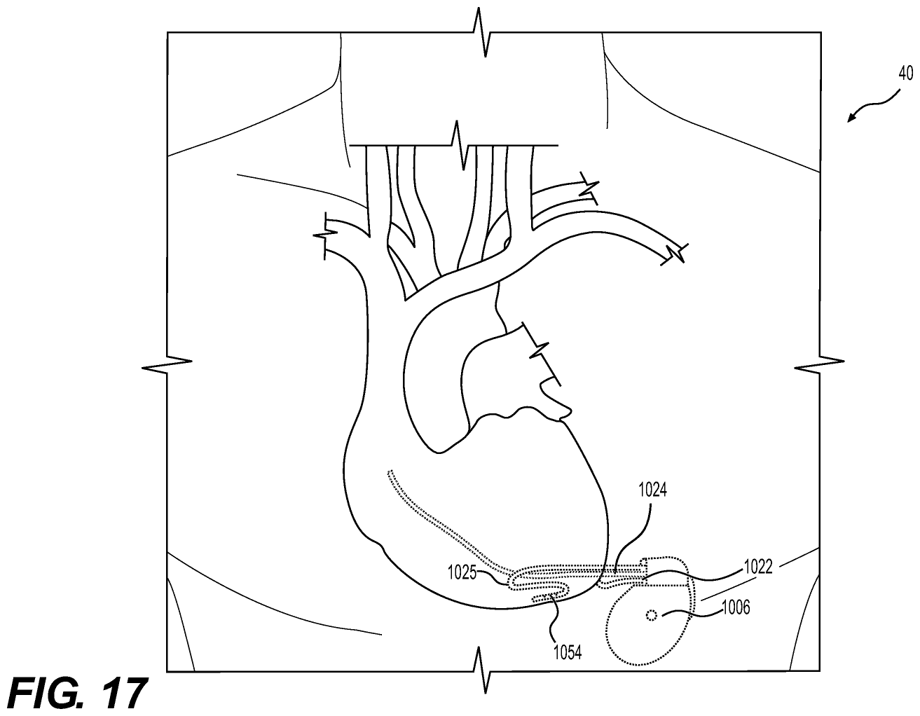

FIG. 17 illustrates another exemplary system 40 that is at least partially epicardially implanted and on the outside of the heart. The epicardial positioning of system 40, relative to the heart, may be illustrated via the dotted lines depicting system 40. It is noted that system 40 may be similar to systems 10′ and 10″, apart from the at least partial epicardial implantation of system 40. Like systems 10′ and 10″, system 40 may comprise a generator 1006, which may be similar to generator 506′, 506″, a plurality of leads 1022, 1024, 1025, which may, respectively, be similar to leads 522′, 522″, 524′, 524″, 525′, 525″, and each of the aforementioned leads may include at least one electrode 1054 (other electrodes not shown), which may be similar to any one of electrodes 532, 534, 542, 544, 552, 554.

As discussed above, system 40 may be positioned so leads 1022, 1024, 1025 (and at least electrodes 1054) are epicardially positioned on aspects of the heart to be treated, e.g., the atrium and/or the ventricle. Thus, each of leads 1022, 1024, 1025 may be adjacent to the atrium and/or the ventricle to deliver far field and near field pulses, in accordance with the atrial/ventricular antiarrhythmic therapies according to this disclosure. The manner by which leads 1022, 1024, 1025 are secured onto the heart is not particularly limited, e.g., suturing. The position of therapy generator 1006 is not particularly limited, and may be any suitable position securely holding therapy generator 1006 in place, e.g., the torso. For example, in FIG. 17, therapy generator 1006 may be positioned in a “pocket” formed by a surgeon that is between layers of upper abdominal muscles, and is configured to hold therapy generator 1006 in place.

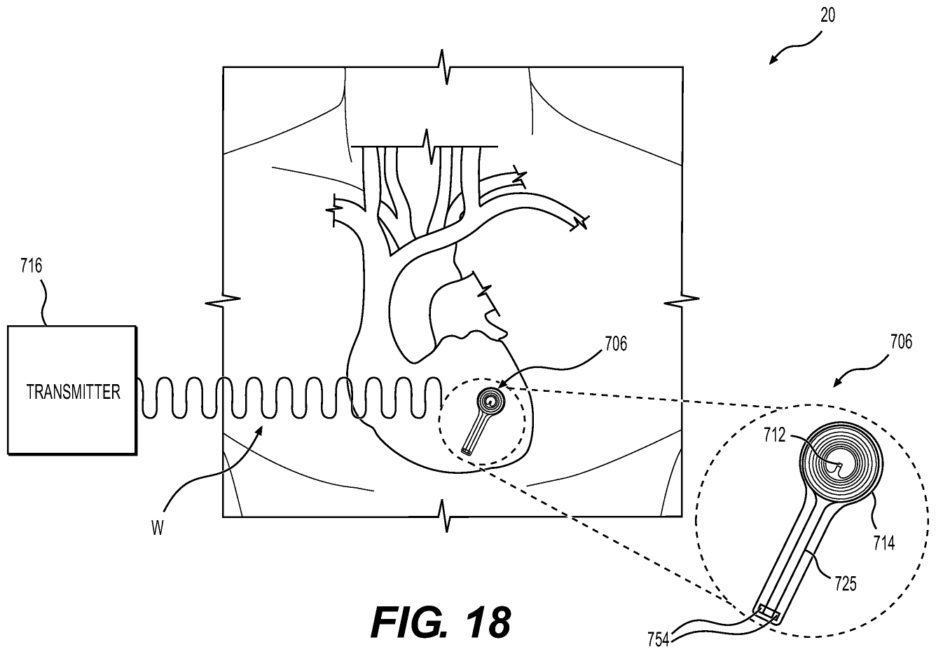

Now referring to FIG. 18, an exemplary leadless and battery-free treatment system 20 is shown. System 20 comprises an external transmitter 716 outside of a patient's body and an implanted leadless treatment device 706 that is in direct contact with the heart of the patient. Transmitter 716 may be in wireless communication with treatment device 706, as represented by line W. As noted above, transmitter 716 may be adapted for wireless delivery of power to device 706 and control of device 706. Transmitter 716 may comprise a transmission coil (not shown) configured for such wireless delivery and communication with device 706. The transmission coil is not particularly limited. Moreover, as previously noted, the external transmitter may comprise sensing circuitry, detection circuitry, control circuitry, and therapy circuitry (not shown), which, collectively, may be configured for the identification of atrial/ventricular arrhythmia and the delivery of atrial/ventricular antiarrhythmic therapies according to this disclosure.

Treatment device 706 may be implanted and positioned on to a region of the heart, as shown in FIG. 18, and may also be fully bioresorbable. Thus, each of the components or features of device 706 discussed below may be of any suitable materials that resorb when exposed to biofluids in a time-controlled manner via metabolic action and hydrolysis. The manner by which device 706 is positioned on to the heart is not particularly limited. For example, in some instances, device 706 may be adhered to the heart via a bioresorbable adhesive between device 706 and the heart. In other instances, device 706 may be sutured on to the heart via a bioresorbable suture, e.g., Ethicon, no. MV-J451-V.

Device 706 comprises a power source (not shown), a diode 712, a receiver 714 adapted for receiving wireless transmissions from transmitter 716, and electrodes 754 in contact with the heart. The power source is not particularly limited, and may be a power harvesting unit, e.g., a dielectric interlayer. Likewise, diode 712 is not particularly limited, and may be a transformer configured to convert waveforms to direct current outputs for stimulation of targeted sites. Diode 712 may be, for example, of a Si NM bioresorbable material. Receiver 714 may include any suitable transmission coil, e.g., a bioresorbable W/Mg coil, in communication with transmitter 716. Electrodes 754 may be any suitable bioresorbable electrodes, e.g., W/Mg electrodes, configured to deliver far field pulses and near field pulses to a targeted region of the heart, in accordance with therapies contemplated throughout this disclosure. Device 706 also comprises a flexible, bioresorbable casing 710, e.g., a PLGA encapsulation, at least partially covering the aforementioned power source, diode 712, receiver 714, and electrodes 754. It is noted that, in certain examples, casing 710 may include an opening exposing electrodes 754 so said electrodes may be in direct contact with a targeted site for the delivery of atrial/ventricular antiarrhythmic therapies according to this disclosure.

Thus, in view of the above, atrial/ventricular antiarrhythmic therapies according to this disclosure may be delivered via subcutaneous means, and also via leadless and/or battery-free modalities as well.

It will be apparent to those skilled in the art that various modifications and variations can be made to the disclosed device without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

1. A system for treating cardiac arrhythmias, the system comprising:
a generator including:
a sensing circuitry configured to evaluate one or more identified signals representative of electrical activity of the heart and detect a tachyarrhythmia;
a control circuitry that is configured to control delivery of a therapy in response to the detected arrhythmia, the therapy including:
a first state of electrical pulses delivered via at least a first electrode, wherein the first set of electrical pulses is configured to destabilize and/or terminate a reentry associated with the arrhythmia; and
a first lead coupled to the generator, wherein the first lead includes the first electrode.

2-30. (canceled)
